(19) United States
(12) Patent Application Publication
(10) Pub. No.: US 2007/0190546 A1
(43) Pub. Date: Aug. 16, 2007

(54) Methods and compositions for sequencing a nucleic acid

(76) Inventors: Suhaib M. Siddiqi, Burlington, MA (US); Philip R. Buzby, Brockton, MA (US); Noubar B. Afeyan, Lexington, MA (US); David R. Liu, Lexington, MA (US)

Correspondence Address: SUGHRUE MION, PLLC, 401 CASTRO STREET, SUITE 220, MOUNTAIN VIEW, CA, 94041-2007, US

(21) Appl. No.: 11/603,945

(22) Filed: Nov. 22, 2006

Related U.S. Application Data
(63) Continuation-in-part of application No. 11/286,626, filed on Nov. 22, 2005.
(63) Continuation-in-part of application No. 11/295,406, filed on Dec. 5, 2005.

(51) Int. Cl. C12Q 1/68; C07H 19/04

(52) U.S. Cl. 435/6; 536/26.1; 536/25.32

(57) ABSTRACT

The invention provides a family of nucleotide analogs useful in sequencing nucleic acids containing a homopolymer region comprising, for example, two or more base repeats, and to sequencing methods using such nucleotide analogs.

### RELATED APPLICATIONS

This application is a continuation-in-part of U.S. Ser. No. 11/286,626 filed Nov. 22, 2005, and is a continuation-in-part of U.S. Ser. No. 11/295,406, filed Dec. 5, 2005, the entire disclosures of which are incorporated by reference herein.

### FIELD OF THE INVENTION

The invention relates to nucleotide analogs and methods for sequencing a nucleic acid using the nucleotide analogs.

### BACKGROUND

Nucleic acid sequencing-by-synthesis has the potential to revolutionize the understanding of biological structure and function. Traditional sequencing technologies rely on amplification of sample-based nucleic acids and/or the use of electrophoretic gels in order to obtain sequence information. More recently, single molecule sequencing has been proposed as a way to obtain high-throughput sequence information that is not subject to amplification bias. See, Braslavsky, Proc. Natl. Acad. Sci. USA 100: 3960-64 (2003).

Sequencing-by-synthesis involves the template-dependent addition of nucleotides to a support-bound template/primer duplex. The added nucleotides are labeled in a manner such that their incorporation into the primer can be detected. A challenge that has arisen in single molecule sequencing involves the ability to sequence through homopolymer regions (i.e., portions of the template that contain-consecutive identical nucleotides). Often the number of bases present in a homopolymer region is important from the point of view of genetic function. As most polymerases used in sequencing-by-synthesis reactions are highly-processive, they tend to add bases continuously as the polymerase traverses a homopolymer region. Most detectable labels used in sequencing reactions do not discriminate between more than two consecutive incorporations. Thus, a homopolymer region will be reported as a single, or sometimes a double, incorporation without the resolution necessary to determine the exact number of bases present in the homopolymer.

A solution to the problem of determining the number of bases present in a homopolymer is proposed in co-owned, co-pending U.S. Patent Application Publication No. US2005/0100932. That method involves controlling the kinetics of the incorporation reaction such that, on average, only a predetermined number of bases are incorporated in any given reaction cycle. The present invention provides an alternative solution to this problem.

### SUMMARY OF THE INVENTION

The invention provides methods and compositions that allow the introduction of a single base at a time in a template-dependent sequencing reaction. The invention allows template-dependent sequencing-by-synthesis through all regions of a target nucleic acid, including homopolymer regions. Thus, the invention also allows for the determination of the number of nucleotides present in a homopolymer region.

The invention contemplates introducing an inhibitor of second nucleotide incorporation in proximity to the active site of incorporation of a first nucleotide. Accordingly, the invention contemplates proximity inhibition in which the concentration of an inhibitor is increased in proximity to the active site of the polymerase, such that a single nucleotide is incorporated but subsequent incorporation is prevented until the inhibition is released. The invention contemplates a number of mechanisms for creating proximity inhibition as discussed below in detail. One mechanism is to couple an inhibitor to a nucleic acid multimer that hybridizes in proximity to the site of base incorporation so as to allow a first base incorporation into a primer portion of a template/primer duplex, but to inhibit any subsequent incorporation until such inhibition has been removed. The inhibitor may also be coupled to the enzyme, to a protein (e.g., an antibody or ligand), or may be linked or “tethered” to the nucleotide to be incorporated.

In one aspect, the invention provides a family of nucleotide analogs, each having a reversible inhibitor or blocker that allow the incorporation of only one nucleotide per addition cycle in a template-dependent sequencing-by-synthesis reaction. The compositions described herein are useful in any sequencing reaction, but are especially useful in single molecule sequencing-by-synthesis reactions. Single molecule reactions are those in which the duplex to which nucleotides are added is individually optically resolvable.

In general, a nucleotide analog of the invention comprises a blocker that is tethered to a nucleotide to be incorporated in a template-dependent sequencing-by-synthesis reaction. The linkage between the nucleotide to be incorporated and the blocker preferably is cleavable so that the blocker can be removed after incorporation of the proper base-paired nucleotide. The blocker portion can be a specific inhibitor or non-specific a non-specific inhibitor of second nucleotide incorporation. In non-specific inhibition, a nucleotide to be incorporated in a sequencing-by-synthesis reaction is linked to a moiety that sterically hinders incorporation of a subsequent nucleotide. In specific inhibition, the blocker is itself a competitive inhibitor of polymerase-catalyzed nucleotide addition. In one embodiment, the inhibitor is a nucleotide that is itself unincorporated but that blocks incorporation downstream of the next complementary nucleotide. In one preferred embodiment, a specific blocker comprises a nucleotide to be incorporated, a lipophilic portion, a mono, di, or triphosphate, and a non-incorporatable deoxyribose or ribose portion.

A tether or linker between the nucleotide to be incorporated and the blocker is from about 4 to about 50 atoms in length. Preferably the linker comprises a lipophilic portion. The linker can also comprise a triple bond or a trans double bond proximal to the base to be incorporated. Finally, the linker contains a cleavable linkage that allows removal of the blocking portion of the molecule.

The base portion of the nucleotide to be incorporated is selected from the standard Watson-Crick bases and their analogs and variants. In the case of the specific inhibitor, the base of the blocking nucleotide is also selected from the standard Watson-Crick bases and their analogs and variants. The incorporated nucleotide and blocking nucleotide can be the same or different. Ideally, the blocking nucleotide is one that is not normally incorporated by a polymerase, such as a nucleotide monophosphate or diphosphate, or one that is lacking the phosphate portion normally attached at the C5′ carbon of the sugar, as shown below.

In a specific embodiment, the invention provides a nucleotide analog comprising a nucleotide to be incorporated linked to a blocking nucleotide comprising a traditional Watson-Crick base (adenine, guanosine, cytosine, thymidine, or uridine), a sugar for example, a ribose or deoxy ribose sugar, and at least one phosphate.

Preferred analogs of the invention comprise an optically-detectable label, for example, a fluorescent label. Labels can be attached to the nucleotide analogs at any position using conventional chemistries such that the label is removed from the incorporated base upon cleavage of the cleavable linker. Examples of useful labels are described in more detail below.

The invention also provides methods for sequencing nucleic acids. In certain methods, a nucleic acid duplex, comprising a template and a primer, is positioned on a surface such that the duplex is individually optically resolvable. A sequencing-by-synthesis reaction is performed under conditions to permit addition of the labeled nucleotide analog to the primer while preventing another nucleotide or nucleotide analog from being added immediately downstream. After incorporation has been detected, inhibition is removed to permit another nucleotide to be added to the primer. Methods of the invention allow detection and counting of consecutive nucleotides in a template homopolymer region.

Specific structures and synthetic pathways are shown below in the detailed description of the invention.

### BRIEF DESCRIPTION OF THE DRAWINGS

FIG. 1 is a schematic representation of reaction scheme for making a first exemplary nucleotide analog of the invention.

FIG. 2 is a schematic representation of reaction scheme for making a second exemplary nucleotide analog of the invention.

FIG. 3 is a schematic representation of reaction scheme for making a third exemplary nucleotide analog of the invention.

### DETAILED DESCRIPTION OF THE INVENTION

The invention provides sequencing-by-synthesis methods for inhibiting second nucleotide (N+1; N being the first base addition) addition to a primer portion of a template/primer duplex. In one embodiment, inhibition of N+1 incorporation is accomplished by increasing the local concentration of an inhibitor that may be present in an overall concentration that is insufficient to provide general incorporation inhibition. In one aspect, the invention provides nucleic acid analogs and methods of using such analogs in template-dependent sequencing-by-synthesis. Analogs of the invention comprise a blocking group that allows the addition of a single nucleotide to a primer portion of a template/primer duplex in a template-dependent reaction. Analogs of the invention comprise a cleavable linker that allows removal of the blocking group in order to permit subsequent (N+1) base addition to the primer. Use of the nucleotide analogs of the invention permits precise sequencing of homopolymer regions and allows the the determination of the number of nucleotides present in such a region.

Preferred analogs of the invention comprise a nucleotide or nucleotide analog to be incorporated linked to a blocker. The blocker may be a bulky steric inhibitor or an unincorporated nucleotide or nucleotide analog linked via a cleavable linker containing, for example, a lipophilic or hydrophilic region. Specific examples of these analogs are provided below for illustrative purpose and in order to demonstrate methods of synthesis. However, the skilled artisan will appreciate that numerous variations are possible, consistent with the scope of the appended claims.

I. Nucleotide Analogs

Nucleotide analogs of the invention have the generalized structure of Formula I or Formula II.

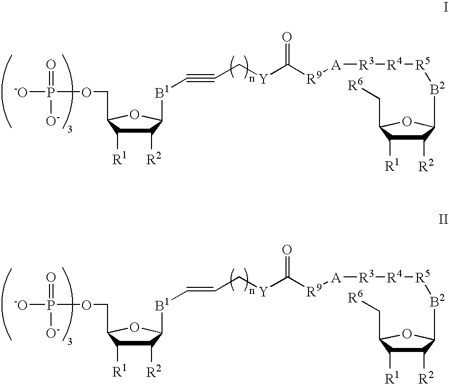

The bases B¹ and B² can each independently be a purine, a pyrimidine, a purine or pyrimidine analog, a bulky group (e.g., a dye, biotin, a bead, or other large molecule). In a preferred embodiment, B¹ and B² are each independently selected from adenine, cytosine, guanine, thymine, uracil, or hypoxanthine. The B¹ and B² groups can also each independently be, for example, naturally-occurring and synthetic derivatives of a base, including pyrazolo[3,4-d]pyrimidines, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-propynyl uracil and cytosine, 6-azo uracil, cytosine and thymine, 5-uracil (pseudouracil), 4-thiouracil, 8-halo (e.g., 8-bromo), 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, deazaguanine, 7-deazaguanine, 3-deazaguanine, deazaadenine, 7-deazaadenine, 3-deazaadenine, pyrazolo[3,4-d]pyrimidine, imidazo[1,5-a]1,3,5 triazinones, 9-deazapurines, imidazo[4,5-d]pyrazines, thiazolo[4,5-d]pyrimidines, pyrazin-2-ones, 1,2,4-triazine, pyridazine; and 1,3,5 triazine.

The nucleotide analogs described herein permit template-dependent incorporation of a single nucleotide. The term base pair encompasses not only the standard AT, AU or GC base pairs, but also base pairs formed between nucleotides and/or nucleotide analogs comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the nucleotide analog inosine and adenine, cytosine or uracil.

In a particular embodiment, the double bond represented by 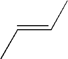
in Formula II is in a trans configuration.

R¹ and R², at each occurrence, independently are selected from the group consisting of OH, H, I, NH₂, and N₃.

The number n, at each occurrence, is independently an integer from 0 to 50. In a preferred embodiment, n is 1 to 4.

Y is selected from the group consisting of NR′, O, S, CH₂, and a bond, wherein R′ is selected from the group consisting of H, alkyl, alkenyl, and alkynyl. Alkyl moieties include saturated aliphatic groups, including straight-chain alkyl groups, branched-chain alkyl groups, cycloalkyl(alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure. The term “alkyl” also includes halosubstituted alkyls. Moreover, the term “alkyl” (or “lower alkyl”) includes “substituted alkyls”, which refers to alkyl moieties having substituents replacing a hydrogen on one or more carbons of the hydrocarbon backbone. The terms “alkenyl” and “alkynyl” refer to unsaturated aliphatic groups analogous in length and possible substitution to the alkyls described above, but that contain at least one double or triple bond respectively.

Where Y is an oxygen, the resulting linker, when cleaved, leaves an exceptionally short “scar” or chemical modification on the incorporated base. The resulting scar is unreactive and does not need to be chemically neutralized. This increases the ease with which a subsequent base can be incorporated. An example of such an analog (using a Cy5 blocker) and the “short scar” elimination is shown below.

Capless dUTP-Cy5

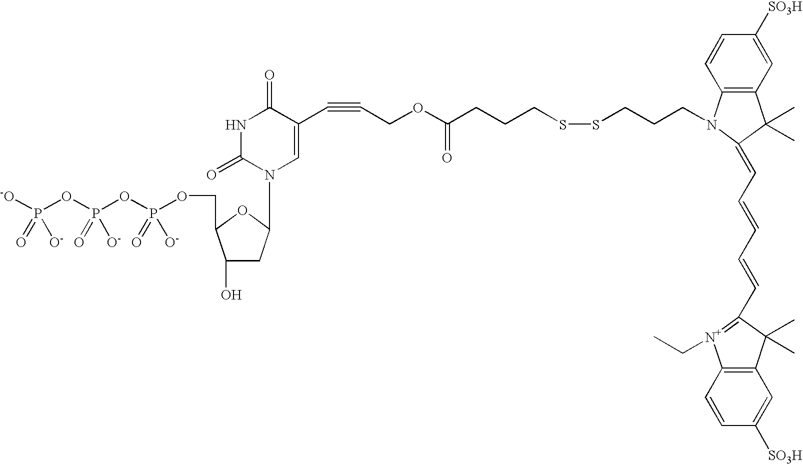

An example of a short scar elimination is set forth below

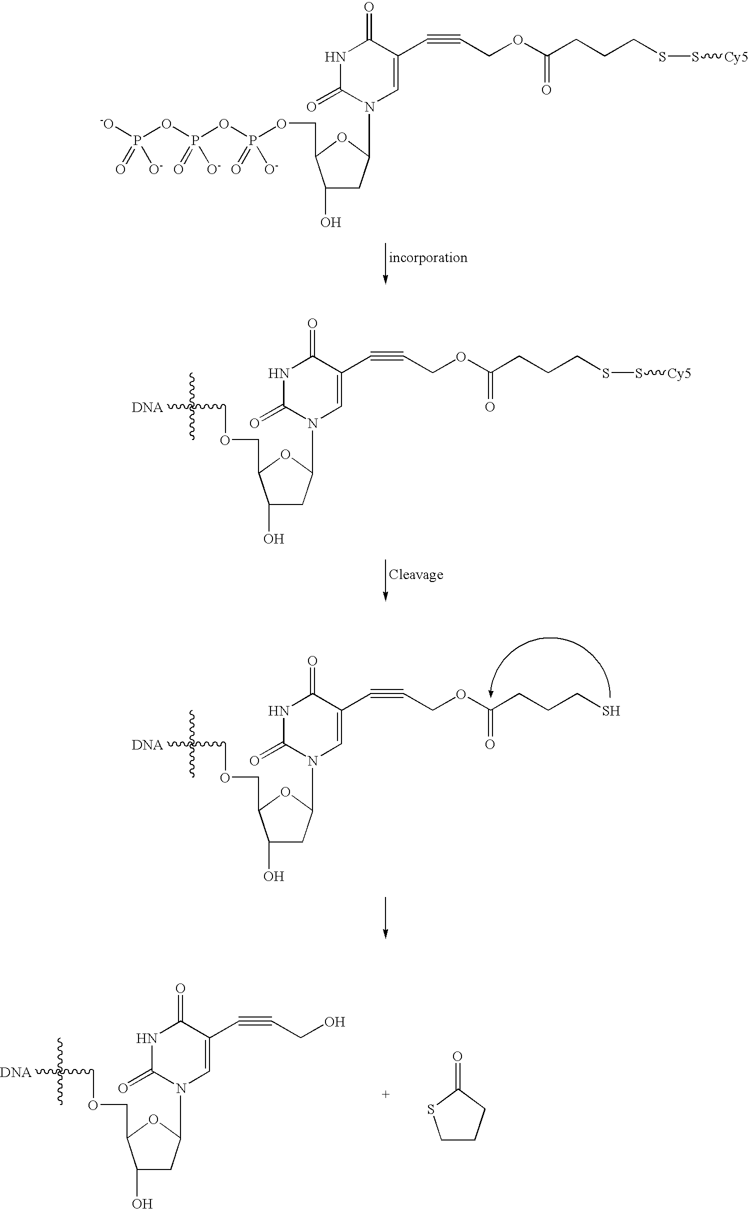

The moiety A is selected from the group consisting of —S—S—, an ester, and an amido group. The term “amido” is art recognized as an amino-substituted carbonyl group.

R³ is selected from the group consisting of: 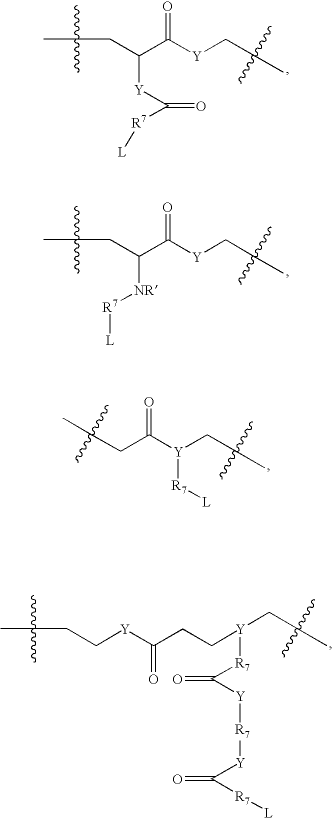
alkyl, and a bond.

R⁴ is selected from the group consisting of alkyl, alkenyl, alkynyl, ether, and a bond. An “ether” may include two hydrocarbons covalently linked by an oxygen. In one embodiment, R⁴ may be a lipophilic moiety. Preferably, R⁴ is glycol ether.

R⁵ is selected from the group consisting of: 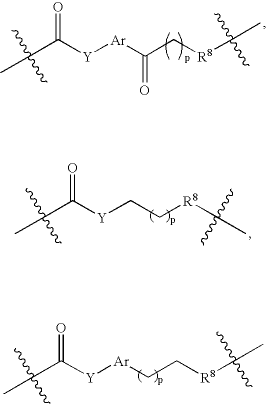
alkyl, alkenyl, and a bond, and where p, at each occurrence, independently is an integer from 0 to 50.

Ar represents an aryl moiety. The term “aryl” refers to 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “heteroaryl” or “heteroaromatics.” The aromatic ring may be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, aryls and/or heterocyclyls. In preferred embodiments, Ar may be phenyl or an aromatic acid.

R⁶ may be any moiety, e.g. a phosphoryl moiety. In some embodiments, R⁶ is selected from the group consisting of: 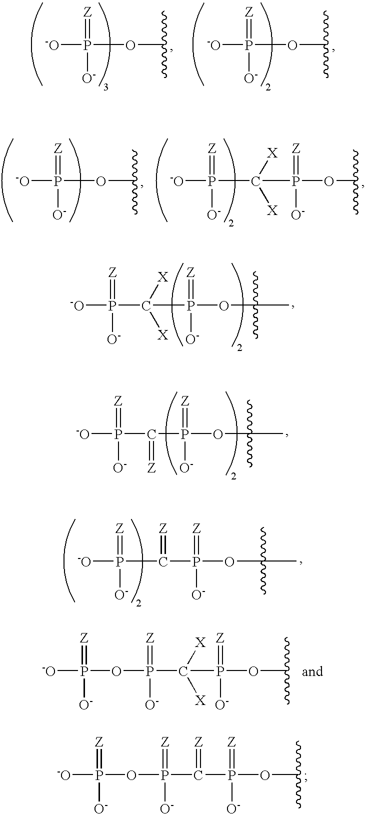
wherein Z, at each occurrence, independently is O or S. X represents H or a halogen, for example, a fluorine, chlorine, bromine or iodine. A preferred halogen is fluorine. In other embodiments, R⁶ may be any moiety with the proviso that R⁶ is not

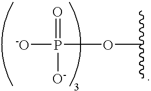

R⁷ may be an alkyl or a bond. R⁸ is selected from S, alkyl, alkenyl, alkynyl, or NR′. R⁹ is selected from NR′, O, S, and —(CH₂)ₘ—, where m is independently an integer from 0 to 50. For example, m may be 0, 1, 2, 3, or 4. In a particular embodiment, n is 4 and m is 3.

L is a label, for example, an optically-detectable label. A variety of optical labels can be used in the practice of the invention and include, for example, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodarnine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cyanine-3 (Cy3); Cyanine-5 (Cy5); Cyanine-5.5 (Cy5.5), Cyanine-7 (Cy7); IRD 700; IRD 800; La Jolta Blue; phthalo cyanine; and naphthalo cyanine.

Preferred labels are fluorescent dyes, such as Cy5 and Cy3. Labels other than fluorescent labels are contemplated by the invention, including other optically-detectable labels. Labels can be attached to the nucleotide analogs of the invention at any position using standard chemistries such that the label can be removed from the incorporated base upon cleavage of the cleavable linker.

For example, when R³ of Formula I or II, is alkyl or a bond, L is covalently bonded to R¹, R², R⁵, R⁶ or B². For example, L may be covalently attached to R¹ or R² via an amide linkage, for example, —CH₂—S—S—CH₂—CH₂—NHCO. L may alternatively be covalently attached to R⁵ or R⁶ via an amide bond.

One exemplary nucleotide analog of the invention is represented as: 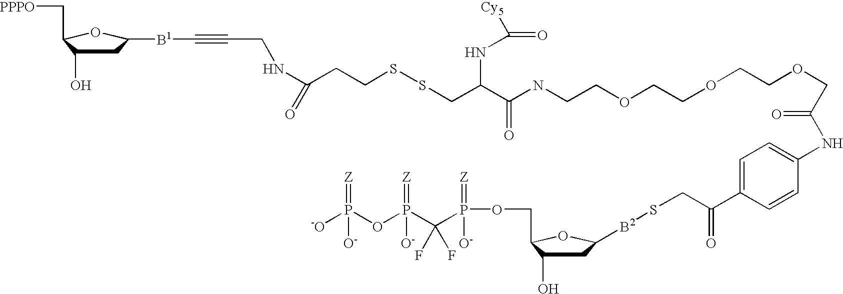
wherein PPPO— is 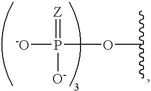
where Z, at each occurrence, independently can be an oxygen or sulfur.

The nucleotide analogs of the invention may also be represented by Formula III: 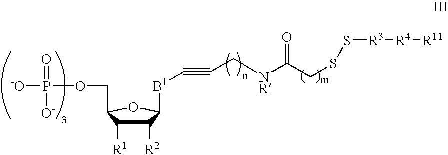
wherein, R¹¹ of Formula III is selected from the group consisting of:

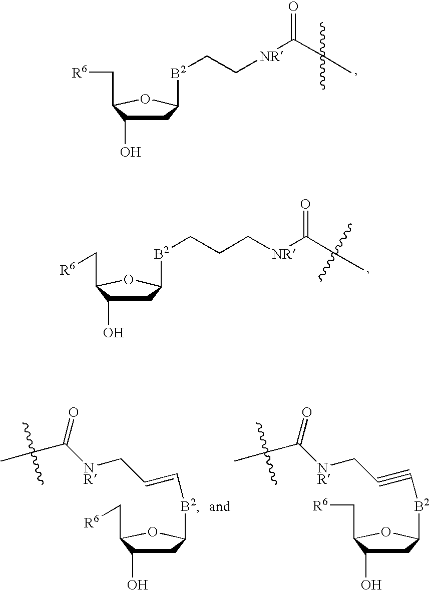

The moieties B¹, B², Y, R′, Z, L, R¹, R², R³, R⁴, R⁶, R⁷, m and n of Formula III are as defined above.

In a particular embodiment, the nucleotide analogs of the invention are selected from the group consisting of:

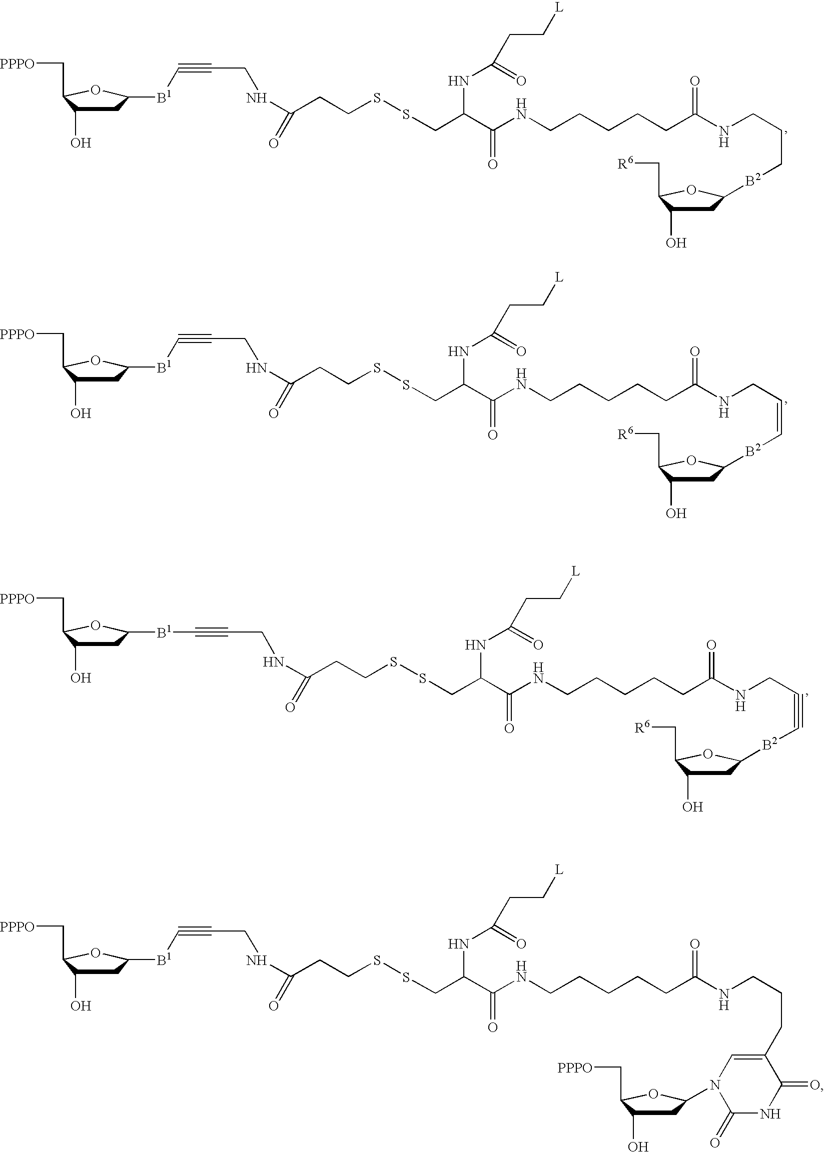

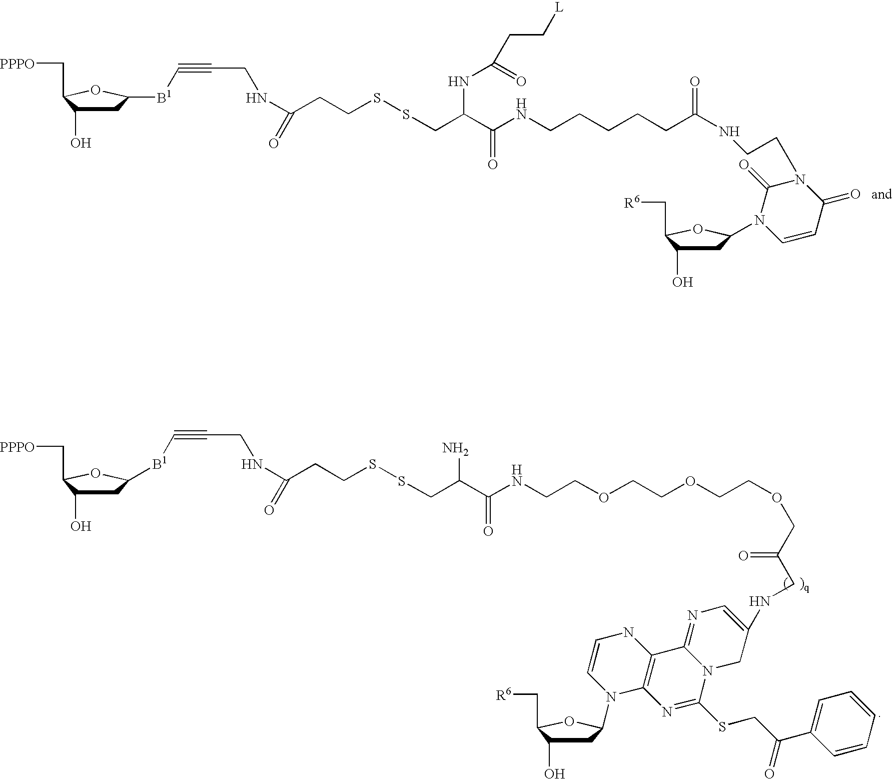

In each embodiment, PPPO— is 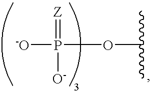
wherein Z, at each occurrence, independently is oxygen or sulfur, and B¹, B², R⁶, and L are as defined above, and q is an integer from 1 to 50.

Another exemplary nucleotide of the invention is the nucleotide analog of Formula IV or Formula V:

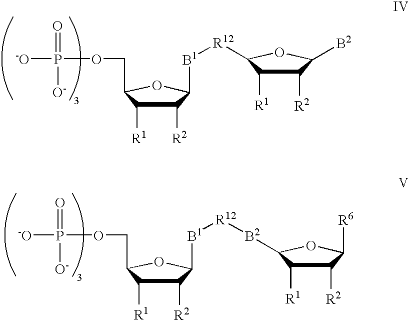

wherein B¹, B², R¹, R² are as defined above; R¹² represents a moiety comprising a cleavable linker. In certain embodiments, R⁶ may represent any moiety with the proviso that R⁶ is not

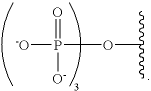

In other embodiments, R⁶ may be as defined above. R¹² may comprise an alkynl moiety bound to B¹. In an embodiment, R¹² may comprise an alkynl moiety bound to B². R¹² may comprise a moiety selected from the group consisting of —S—S—, an ester, and an amido group.

II. Template-Directed Sequencing By Synthesis

As discussed above, the invention provides improved methods for sequencing a nucleic acid containing a homopolymer region. The method comprises exposing a nucleic acid template/primer duplex to (i) a polymerase which catalyzes nucleotide addition to the primer, and (ii) a labeled nucleotide analog comprising a first nucleotide or a first nucleotide analog covalently bonded through a linker to a blocker under conditions that permit the polymerase to add the labeled nucleotide analog to the primer at a position complementary to the first base in the template while preventing another nucleotide or nucleotide analog from being added to the primer at a position complementary to the next downstream base. After the exposing step, the nucleotide analog incorporated into the primer is detected. The blocker is removed to permit other nucleotides to be incorporated into the primer. It is contemplated that the label, for example, one of the optically detectable labels described herein, can be removed at the same time as the blocker.

Any of the nucleotide analogs described herein can be used in this type of sequencing protocol. In certain embodiments, however, the linker is covalently attached to the base of the first nucleotide or first nucleotide analog and to the base of the blocking nucleotide or blocking nucleotide analog. In certain other embodiments, the linker is from about 4 to about 50 atoms in length, or from about 15 to about 15 atoms in length. In other embodiments, the linker is covalently attached to the first nucleotide or first nucleotide analog via an alkynyl group or via an alkenyl group containing a double bond in a trans configuration.

The following sections discuss general considerations for nucleic acid sequencing, for example, template considerations, polymerases useful in sequencing-by-synthesis, choice of surfaces, reaction conditions, signal detection and analysis.

Nucleic Acid Templates

Nucleic acid templates include deoxyribonucleic acid (DNA) and/or ribonucleic acid (RNA). Nucleic acid templates can be synthetic or derived from naturally occurring sources. In one embodiment, nucleic acid template molecules are isolated from a biological sample containing a variety of other components, such as proteins, lipids and non-template nucleic acids. Nucleic acid template molecules can be obtained from any cellular material, obtained from an animal, plant, bacterium, fungus, or any other cellular organism. Biological samples for use in the present invention include viral particles or preparations. Nucleic acid template molecules can be obtained directly from an organism or from a biological sample obtained from an organism, e.g., from blood, urine, cerebrospinal fluid, seminal fluid, saliva, sputum, stool and tissue. Any tissue or body fluid specimen may be used as a source for nucleic acid for use in the invention. Nucleic acid template molecules can also be isolated from cultured cells, such as a primary cell culture or a cell line. The cells or tissues from which template nucleic acids are obtained can be infected with a virus or other intracellular pathogen. A sample can also be total RNA extracted from a biological specimen, a cDNA library, viral, or genomic DNA.

Nucleic acid obtained from biological samples typically is fragmented to produce suitable fragments for analysis. In one embodiment, nucleic acid from a biological sample is fragmented by sonication. Nucleic acid template molecules can be obtained as described in U.S. Patent Application Publication Number US2002/0190663 A1, published Oct. 9, 2003. Generally, nucleic acid can be extracted from a biological sample by a variety of techniques such as those described by Maniatis, et al., Molecular Cloning: A Laboratory Manual, Cold Spring Harbor, N.Y., pp. 280-281 (1982). Generally, individual nucleic acid template molecules can be from about 5 bases to about 20 kb. Nucleic acid molecules may be single-stranded, double-stranded, or double-stranded with single-stranded regions (for example, stem- and loop-structures).

A biological sample as described herein may be homogenized or fractionated in the presence of a detergent or surfactant. The concentration of the detergent in the buffer may be about 0.05% to about 10.0%. The concentration of the detergent can be up to an amount where the detergent remains soluble in the solution. In a preferred embodiment, the concentration of the detergent is between 0.1% to about 2%. The detergent, particularly a mild one that is nondenaturing, can act to solubilize the sample. Detergents may be ionic or nonionic. Examples of nonionic detergents include triton, such as the Triton® X series (Triton® X-100 t-Oct-C₆H₄—(OCH₂—CH₂)ₓOH, x=9-10, Triton® X-100R, Triton® X-114 x=7-8), octyl glucoside, polyoxyethylene(9)dodecyl ether, digitonin, IGEPAL® CA630 octylphenyl polyethylene glycol, n-octyl-beta-D-glucopyranoside (betaOG), n-dodecyl-beta, Tween® 20 polyethylene glycol sorbitan monolaurate, Tween® 80 polyethylene glycol sorbitan monooleate, polidocanol, n-dodecyl beta-D-maltoside (DDM), NP-40 nonylphenyl polyethylene glycol, C12E8 (octaethylene glycol n-dodecyl monoether), hexaethyleneglycol mono-n-tetradecyl ether (C14EO6), octyl-beta-thioglucopyranoside (octyl thioglucoside, OTG), Emulgen, and polyoxyethylene 10 lauryl ether (C12E10). Examples of ionic detergents (anionic or cationic) include deoxycholate, sodium dodecyl sulfate (SDS), N-lauroylsarcosine, and cetyltrimethylammoniumbromide (CTAB). A zwitterionic reagent may also be used in the purification schemes of the present invention, such as Chaps, zwitterion 3-14, and 3-[(3-cholamidopropyl)dimethylammonio]-1-propanesulf-onate. It is contemplated also that urea may be added with or without another detergent or surfactant.

Lysis or homogenization solutions may further contain other agents, such as reducing agents. Examples of such reducing agents include dithiothreitol (DTT), β-mercaptoethanol, DTE, GSH, cysteine, cysteamine, tricarboxyethyl phosphine (TCEP), or salts of sulfurous acid.

Nucleic Acid Polymerases

Nucleic acid polymerases generally useful in the invention include DNA polymerases, RNA polymerases, reverse transcriptases, and mutant or altered forms of any of the foregoing. DNA polymerases and their properties are described in detail in, among other places, DNA Replication 2nd edition, Kornberg and Baker, W. H. Freeman, New York, N.Y. (1991). Known conventional DNA polymerases useful in the invention include, but are not limited to, Pyrococcus furiosus (Pfu) DNA polymerase (Lundberg et al., 1991, Gene, 108: 1, Stratagene), Pyrococcus woesei (Pwo) DNA polymerase (Hinnisdaels et al., 1996, Biotechniques, 20:186-8, Boehringer Mannheim), Thermus thermophilus (Tth) DNA polymerase (Myers and Gelfand 1991, Biochemistry 30:7661), Bacillus stearothermophilus DNA polymerase (Stenesh and McGowan, 1977, Biochim Biophys Acta 475:32), Thermococcus litoralis (Tli) DNA polymerase (also referred to as Vent™ DNA polymerase, Cariello et al., 1991, Polynucleotides Res, 19: 4193, New England Biolabs), 9°Nm™ DNA polymerase (New England Biolabs), Stoffel fragment, ThermoSequenase® (Amersham Pharmacia Biotech UK), Therminator™ (New England Biolabs), Thermotoga maritima (Tma) DNA polymerase (Diaz and Sabino, 1998 Braz J Med. Res, 31:1239), Thermus aquaticus (Taq) DNA polymerase (Chien et al., 1976, J. Bacteoriol, 127: 1550), DNA polymerase, Pyrococcus kodakaraensis KOD DNA polymerase (Takagi et al., 1997, Appl. Environ. Microbiol. 63:4504), JDF-3 DNA polymerase (from thermococcus sp. JDF-3, Patent application WO 0132887), Pyrococcus GB-D (PGB-D) DNA polymerase (also referred as Deep Vent™ DNA polymerase, Juncosa-Ginesta et al., 1994, Biotechniques, 16:820, New England Biolabs), UlTma DNA polymerase (from thermophile Thermotoga maritima; Diaz and Sabino, 1998 Braz J. Med. Res, 31:1239; PE Applied Biosystems), Tgo DNA polymerase (from thermococcus gorgonarius, Roche Molecular Biochemicals), E. coli DNA polymerase I (Lecomte and Doubleday, 1983, Polynucleotides Res. 11:7505), T7 DNA polymerase (Nordstrom et al., 1981, J Biol. Chem. 256:3112), and archaeal DP1I/DP2 DNA polymerase II (Cann et al., 1998, Proc. Natl. Acad. Sci. USA 95:14250).

Both mesophilic polymerases and thermophilic polymerases are contemplated. Thermophilic DNA polymerases include, but are not limited to, ThennoSequenase®, 9°Nm™, Therminator™, Taq, Tne, Tma, Pfu, Tfl, Tth, Tli, Stoffel fragment, Vent™ and Deep Vent™ DNA polymerase, KOD DNA polymerase, Tgo, JDF-3, and mutants, variants and derivatives thereof. A highly-preferred form of any polymerase is a 3′ exonuclease-deficient mutant.

Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)).

Surfaces

In a preferred embodiment, nucleic acid template molecules are attached to a substrate (also referred to herein as a surface) and subjected to analysis by single molecule sequencing as described herein. Nucleic acid template molecules are attached to the surface such that the template/primer duplexes are individually optically resolvable. Substrates for use in the invention can be two- or three-dimensional and can comprise a planar surface (e.g., a glass slide) or can be shaped. A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites.

Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a nucleic acid. Substrates can include planar arrays or matrices capable of having regions that include populations of template nucleic acids or primers. Examples include nucleoside-derivatized CPG and polystyrene slides; derivatized magnetic slides; polystyrene grafted with polyethylene glycol, and the like.

Substrates are preferably coated to allow optimum optical processing and nucleic acid attachment. Substrates for use in the invention can also be treated to reduce background. Exemplary coatings include epoxides, and derivatized epoxides (e.g., with a binding molecule, such as an oligonucleotide or streptavidin).

Various methods can be used to anchor or immobilize the nucleic acid molecule to the surface of the substrate. The immobilization can be achieved through direct or indirect bonding to the surface. The bonding can be by covalent linkage. See, Joos et al., Analytical Biochemistry 247:96-101, 1997; Oroskar et al., Clin. Chem. 42:1547-1555, 1996; and Khandjian, Mol. Bio. Rep. 11: 107-115, 1986. A preferred attachment is direct amine bonding of a terminal nucleotide of the template or the 5′ end of the primer to an epoxide integrated on the surface. The bonding also can be through non-covalent linkage. For example, biotin-streptavidin (Taylor et al., J. Phys. D. Appl. Phys. 24:1443, 1991) and digoxigenin with anti-digoxigenin (Smith et al., Science 253:1122, 1992) are common tools for anchoring nucleic acids to surfaces and parallels. Alternatively, the attachment can be achieved by anchoring a hydrophobic chain into a lipid monolayer or bilayer. Other methods for known in the art for attaching nucleic acid molecules to substrates also can be used.

Detection

Any detection method can be used that is suitable for the type of label employed. Thus, exemplary detection methods include radioactive detection, optical absorbance detection, e.g., UV-visible absorbance detection, optical emission detection, e.g., fluorescence or chemiluminescence. For example, extended primers can be detected on a substrate by scanning all or portions of each substrate simultaneously or serially, depending on the scanning method used. For fluorescence labeling, selected regions on a substrate may be serially scanned one-by-one or row-by-row using a fluorescence microscope apparatus, such as described in Fodor (U.S. Pat. No. 5,445,934) and Mathies et al. (U.S. Pat. No. 5,091,652). Devices capable of sensing fluorescence from a single molecule include scanning tunneling microscope (siM) and the atomic force microscope (AFM). Hybridization patterns may also be scanned using a CCD camera (e.g., Model TE/CCD512SF, Princeton Instruments, Trenton, N.J.) with suitable optics (Ploem, in Fluorescent and Luminescent Probes for Biological Activity Mason, T. G. Ed., Academic Press, Landon, pp. 1-11 (1993), such as described in Yershov et al., Proc. Natl. Acad. Sci. 93:4913 (1996), or may be imaged by TV monitoring. For radioactive signals, a phosphorimager device can be used (Johnston et al., Electrophoresis, 13:566, 1990; Drmanac et al., Electrophoresis, 13:566, 1992; 1993). Other commercial suppliers of imaging instruments include General Scanning Inc., (Watertown, Mass. on the World Wide Web at genscan.com), Genix Technologies (Waterloo, Ontario, Canada; on the World Wide Web at confocal.com), and Applied Precision Inc. Such detection methods are particularly useful to achieve simultaneous scanning of multiple attached template nucleic acids.

A number of approaches can be used to detect incorporation of fluorescently-labeled nucleotides into a single nucleic acid molecule. Optical setups include near-field scanning microscopy, far-field confocal microscopy, wide-field epi-illumination, light scattering, dark field microscopy, photoconversion, single and/or multiphoton excitation, spectral wavelength discrimination, fluorophor identification, evanescent wave illumination, and total internal reflection fluorescence (TIRF) microscopy. In general, certain methods involve detection of laser-activated fluorescence using a microscope equipped with a camera. Suitable photon detection systems include, but are not limited to, photodiodes and intensified CCD cameras. For example, an intensified charge couple device (ICCD) camera can be used. The use of an ICCD camera to image individual fluorescent dye molecules in a fluid near a surface provides numerous advantages. For example, with an ICCD optical setup, it is possible to acquire a sequence of images (movies) of fluorophores.

Some embodiments of the present invention use TIRF microscopy for imaging. TIRF microscopy uses totally internally reflected excitation light and is well known in the art. See, e g., the World Wide Web at nikon-instruments.jp/eng/page/products/tirf.aspx. In certain embodiments, detection is carried out using evanescent wave illumination and total internal reflection fluorescence microscopy. An evanescent light field can be set up at the surface, for example, to image fluorescently-labeled nucleic acid molecules. When a laser beam is totally reflected at the interface between a liquid and a solid substrate (e.g., a glass), the excitation light beam penetrates only a short distance into the liquid. The optical field does not end abruptly at the reflective interface, but its intensity falls off exponentially with distance. This surface electromagnetic field, called the “evanescent wave”, can selectively excite fluorescent molecules in the liquid near the interface. The thin evanescent optical field at the interface provides low background and facilitates the detection of single molecules with high signal-to-noise ratio at visible wavelengths.

The evanescent field also can image fluorescently-labeled nucleotides upon their incorporation into the attached template/primer complex in the presence of a polymerase. Total internal reflectance fluorescence microscopy is then used to visualize the attached template/primer duplex and/or the incorporated nucleotides with single molecule resolution.

Analysis

Alignment and/or compilation of sequence results obtained from the image stacks produced as generally described above utilizes look-up tables that take into account possible sequences changes (due, e.g., to errors, mutations, etc.). Essentially, sequencing results obtained as described herein are compared to a look-up type table that contains all possible reference sequences plus 1 or 2 base errors.

### EXAMPLES

The invention is further illustrated by the following non-limiting examples, which describe the synthesis of a number of exemplary nucleotide analogs of the invention (Examples 1-3), and their use in nucleic acid sequencing (Example 4).

### Example 1

This example describes the synthesis of the following nucleotide analog, denoted as nucleotide analog 5.

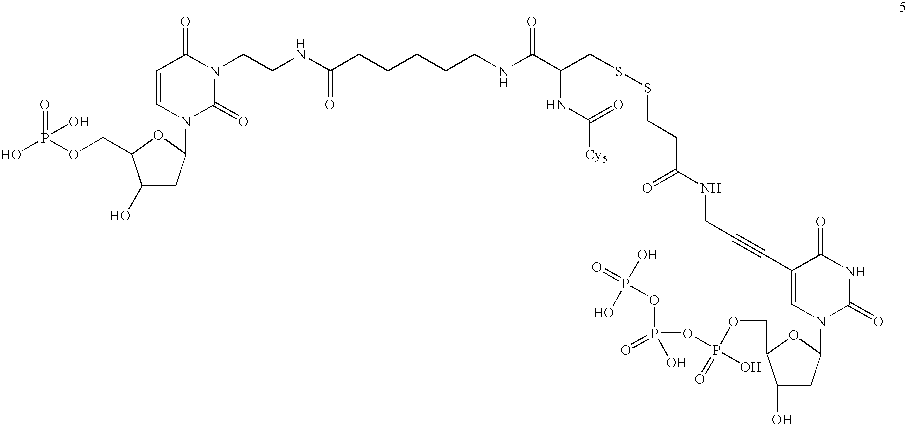

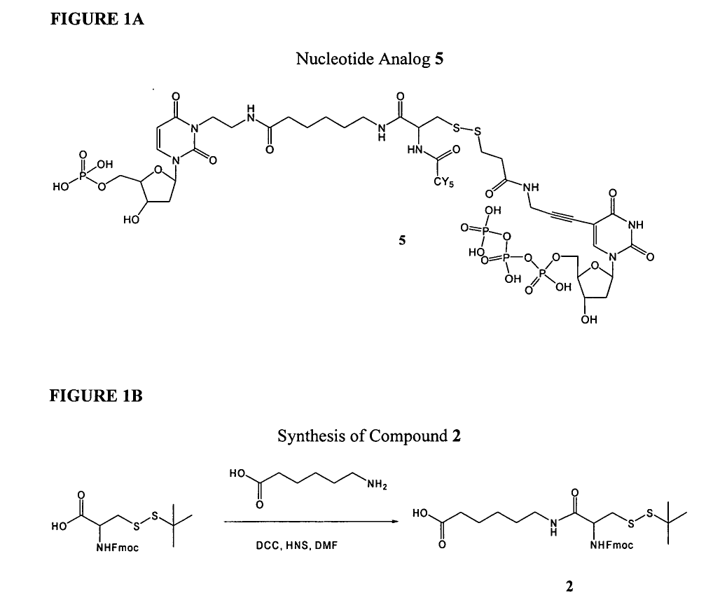

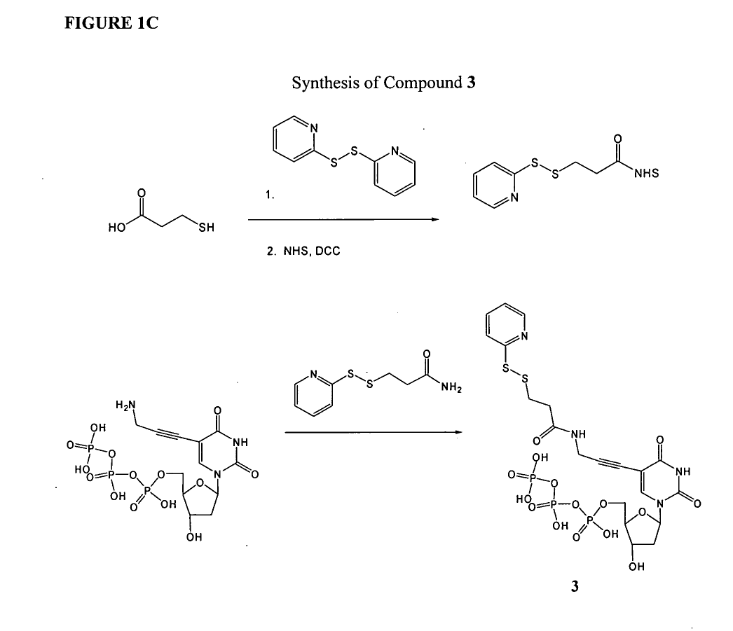

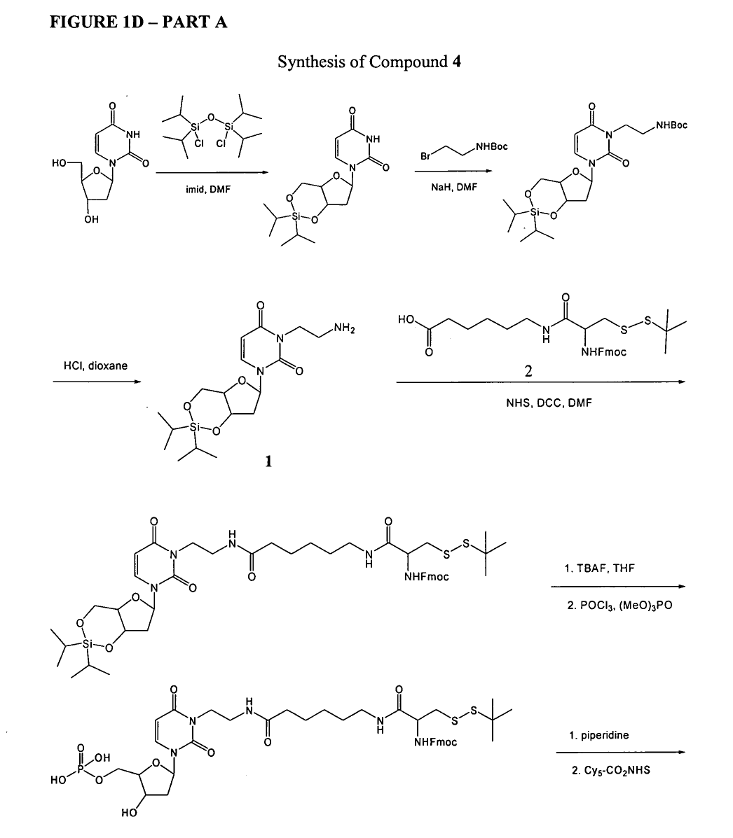

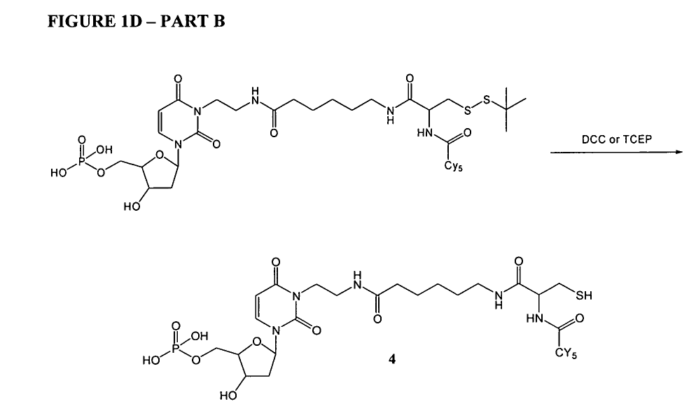

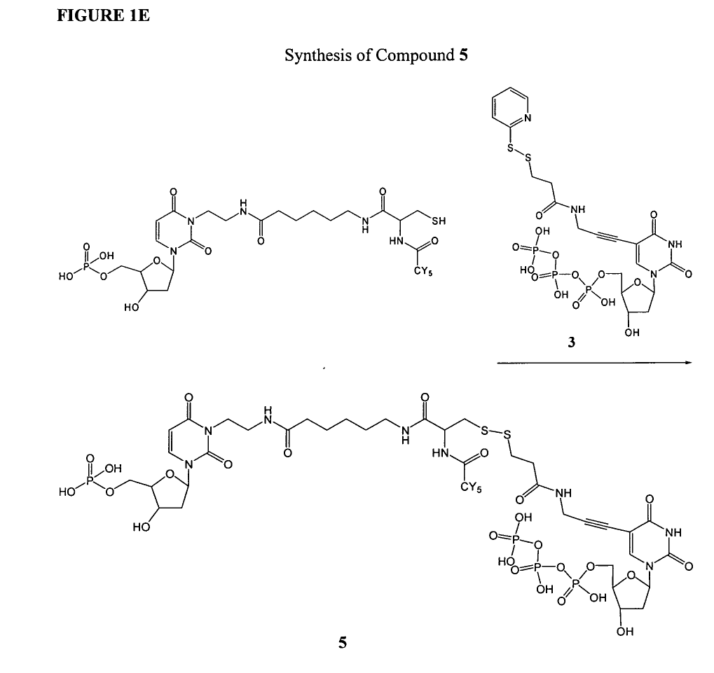

In this example, both bases are uracil, however, it is appreciated that the skilled artisan can make similar nucleotides analogs containing other bases using similar chemistries. The reaction scheme to synthesize nucleotide analog 5 is set forth in FIG. 1. In particular, FIG. 1A depicts nucleotide analog 5, FIG. 1B describes the synthesis of compound 2 (an intermediate in the synthesis of analog 5), FIG. 1B describes the synthesis of compound 3 (an intermediate in the synthesis of analog 5), FIG. 1D describes the synthesis of compounds 1 and 4 intermediates in the synthesis of analog 5), and FIG. 1E describes the synthesis of nucleotide analog 5.

### Example 2

This example describes the synthesis of the nucleotide analog, denoted nucleotide analog 7.

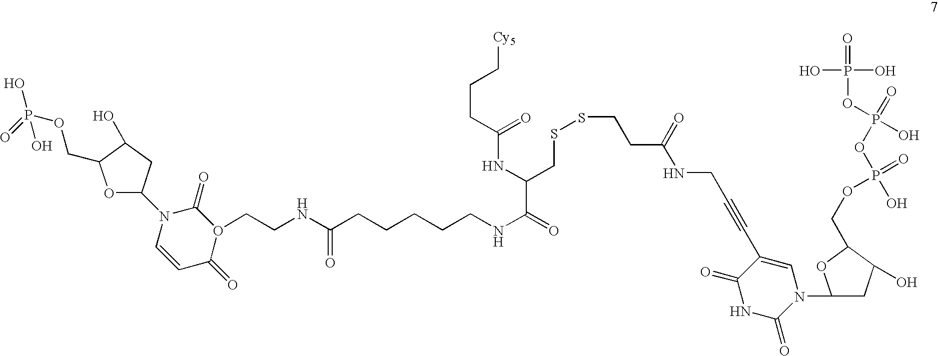

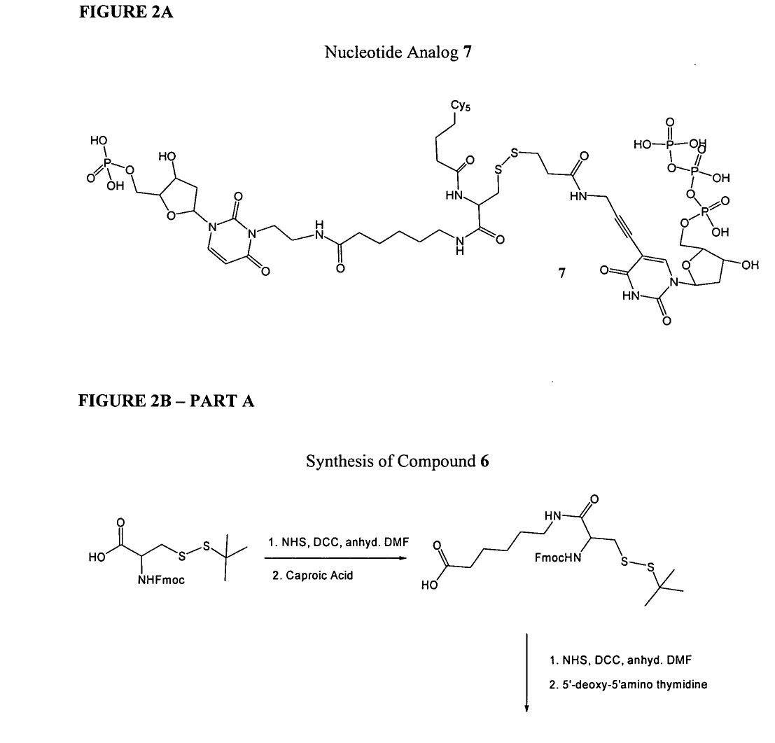

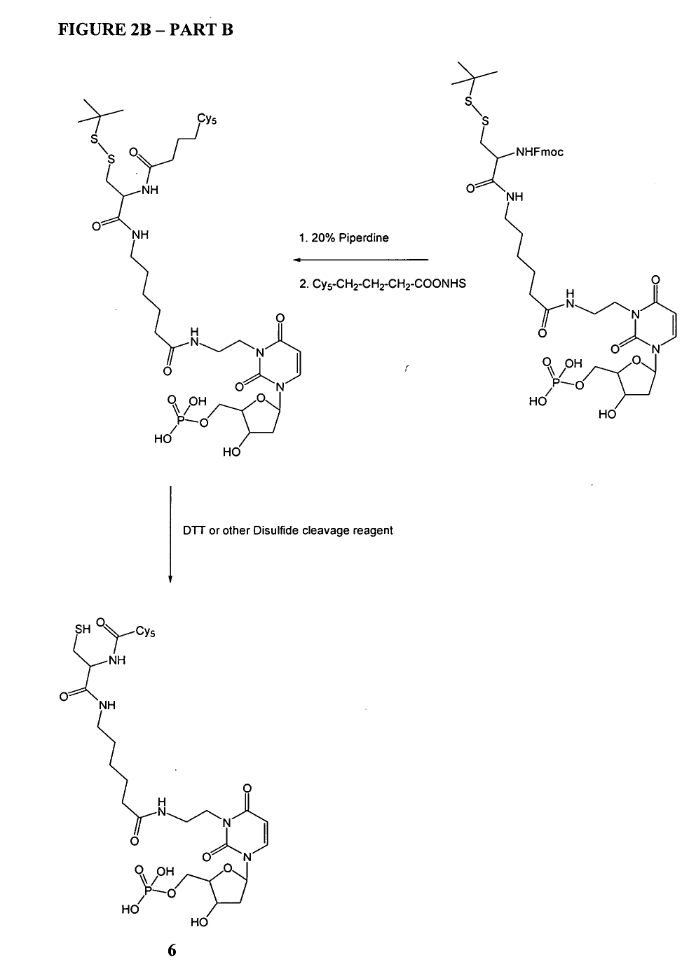

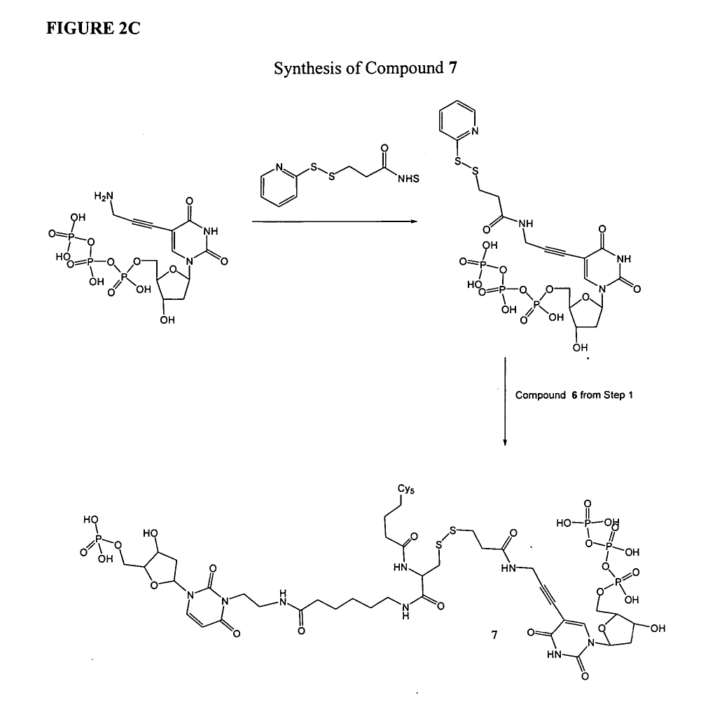

In this example, both bases are uracil, however, it is appreciated that the skilled artisan can make similar nucleotides analogs containing other bases using similar chemistries. The reaction scheme to synthesize nucleotide analog 7 is set forth in FIG. 2. In particular, FIG. 2A shows nucleotide analog 7, FIG. 2B describes the synthesis of compound 6 (an intermediate in the synthesis of nucleotide analog 7), FIG. 2C describes the steps in the synthesis of nucleotide analog 7.

### Example 3

This example describes the synthesis of the nucleotide analog, denoted nucleotide analog 9, where n is 3.

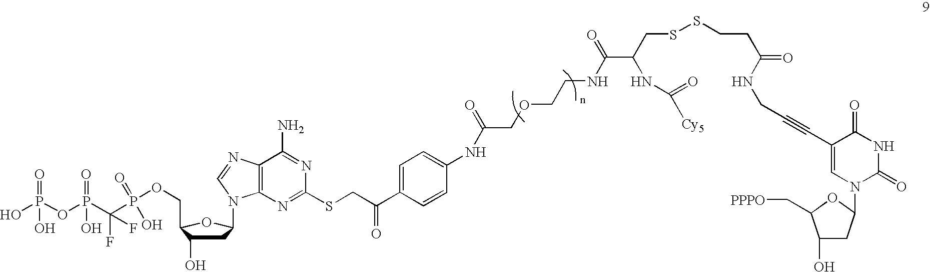

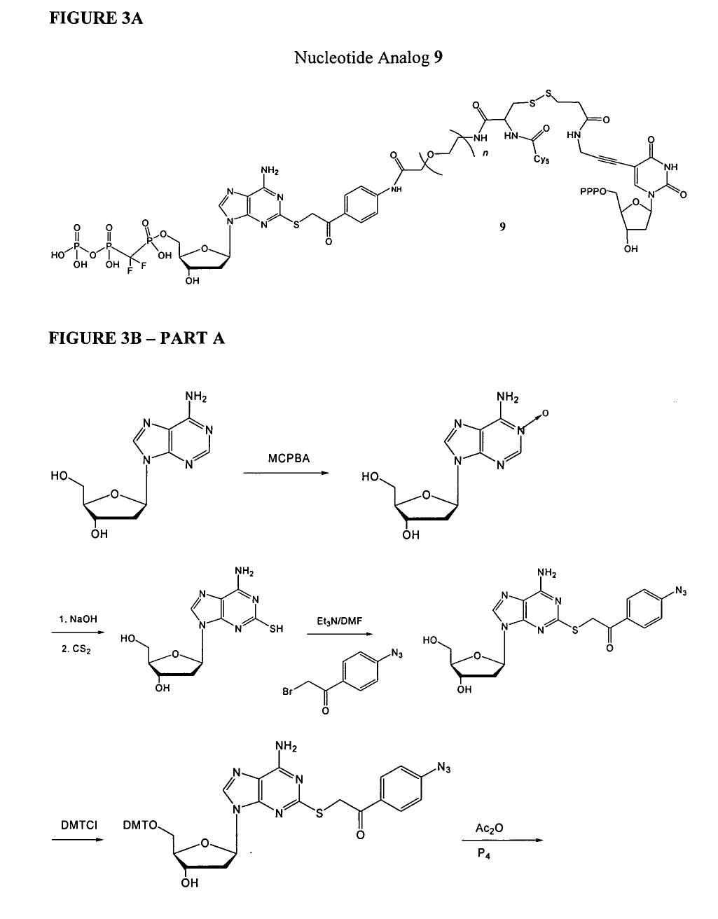

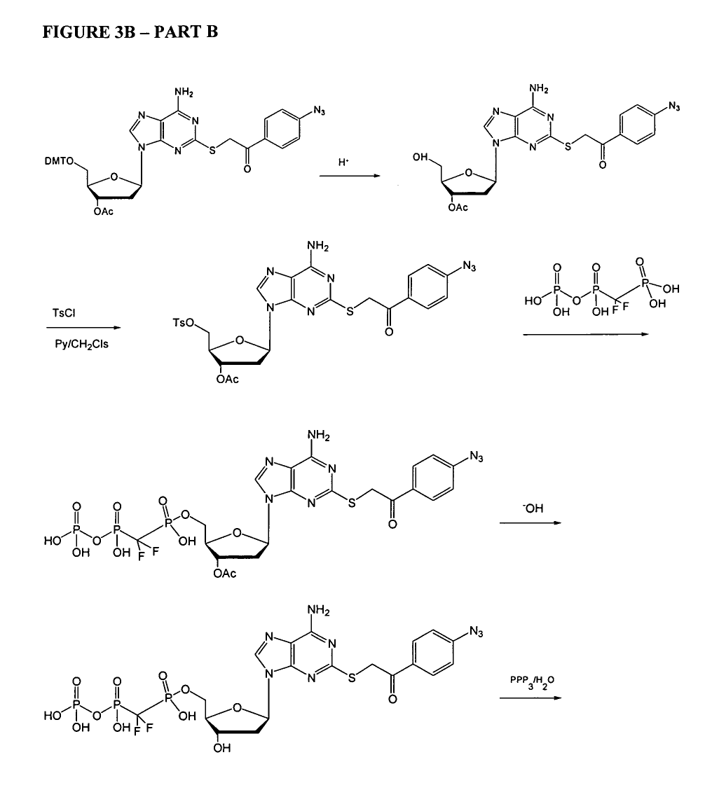

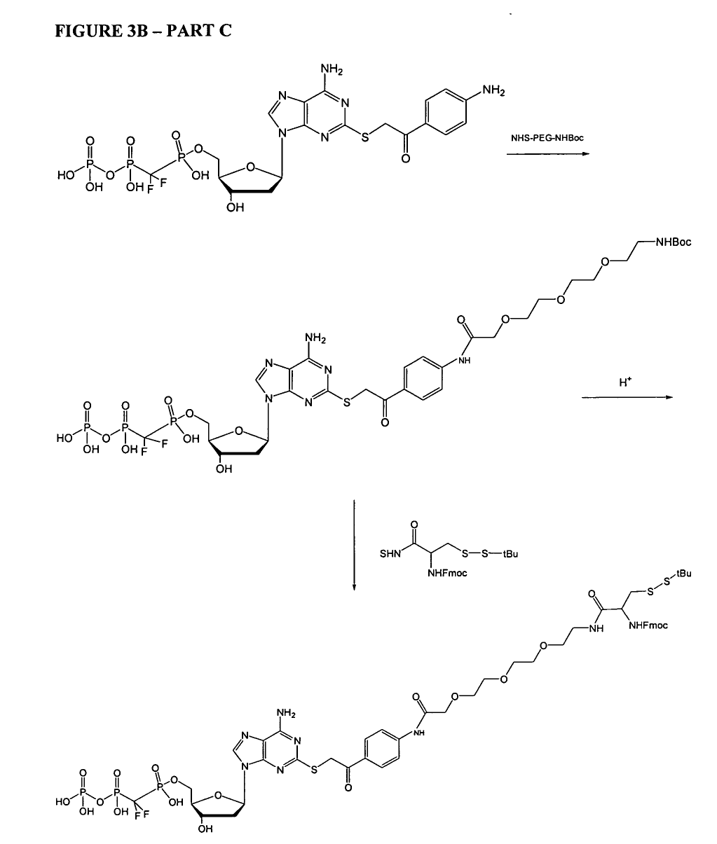

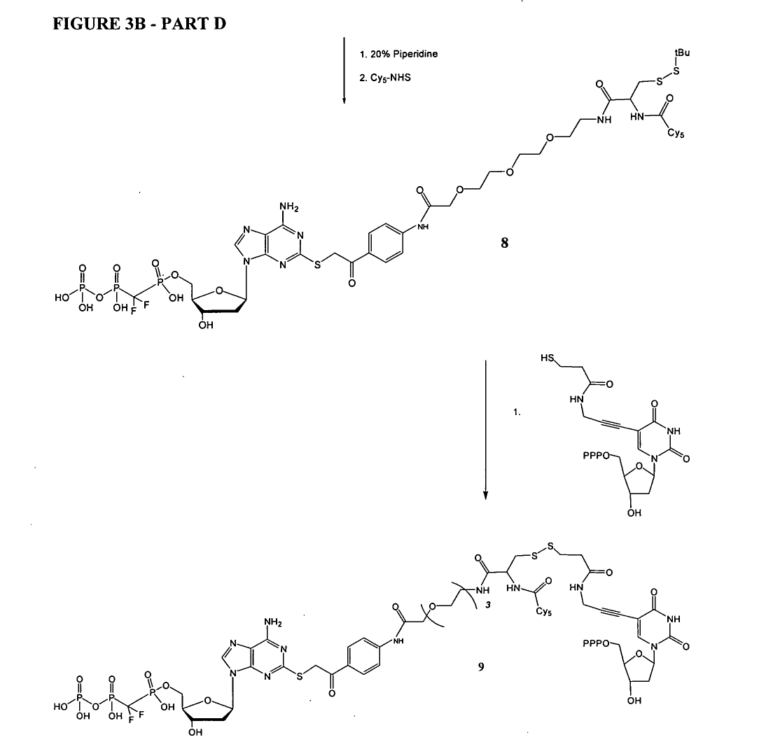

In this example, one base is an adenine while the other base is a uracil. It is appreciated that the skilled artisan can make similar nucleotides analogs containing other bases using similar chemistries. The reaction scheme to synthesize nucleotide analog 9 is set forth in FIG. 3. In particular, FIG. 3A shows nucleotide analog 9, and FIG. 3B shows the steps in the synthesis of nucleotide analog 9.

### Example 4

This example describes a method for sequencing a template nucleic acid using certain nucleotide analogs described herein.

The 7249 nucleotide genome of the bacteriophage M13mp18 is sequenced using analogs and methods of the invention. Purified, single-stranded viral M13mp18 genomic DNA was obtained from New England Biolabs. Approximately 25 μg of M13 DNA was digested to an average fragment size of 40-100 bp with 0.1 U Dnase I (New England Biolabs) for 10 minutes at 37° C. Digested DNA fragment sizes were estimated by running an aliquot of the digestion mixture on a precast denaturing (TBE-Urea) 10% polyacrylamide gel (Novagen) and staining with SYBR Gold (Invitrogen/Molecular Probes). The DNase I-digested genomic DNA was filtered through a YM10 ultrafiltration spin column (Millipore) to remove small digestion products less than about 10 nucleotides. Approximately 20 pmol of the filtered DNase I digest then was polyadenylated with terminal transferase according to known methods (Roychoudhury, R and Wu, R. 1980, Terminal transferase-catalyzed addition of nucleotides to the 3′ termini of DNA. Methods Enzymol. 65(1):43-62.). The average dA tail length was 50±5 nucleotides. Terminal transferase then was used to label the fragments with Cy3-dUTP. Fragments then were terminated with dideoxyTTP (also added using terminal transferase). The resulting fragments were again filtered with a YM10 ultrafiltration spin column to remove free nucleotides and stored in ddH₂O at −20° C.

Epoxide-coated glass slides were prepared for oligo attachment. Epoxide-functionalized 40 mm diameter #1.5 glass cover slips (slides) were obtained from Erie Scientific (Salem, N.H.). The slides were preconditioned by soaking in 3×SSC for 15 minutes at 37° C. Next, a 500 pM aliquot of 5′ aminated template fragments described above are incubated with each slide for 30 minutes at room temperature in a volume of 80 mL. The resulting slides have poly(dT50) template fragments attached by direct amine linkage to the epoxide. The slides are then treated with phosphate (1 M) for 4 hours at room temperature in order to passivate the surface. Slides are then stored in buffer (20 mM Tris, 100 mM NaCl, 0.001% Triton X-100, pH 8.0) until they are used for sequencing.

For sequencing, the slides are placed in a modified FCS2 flow cell (Bioptechs, Butler, Pa.) using a 50 μm thick gasket The flow cell is placed on a movable stage that is part of a high-efficiency fluorescence imaging system built around a Nikon TE-2000 inverted microscope equipped with a total internal reflection (TIR) objective. The slide then is rinsed with HEPES buffer with 100 mM NaCl and equilibrated to a temperature of 50° C. An aliquot of poly(dT50) primer is placed in the flow cell and incubated on the slide for 15 minutes. After incubation, the flow cell is rinsed with 1×SSC/HEPES/0.1% SDS followed by HEPES/NaCl. A passive vacuum apparatus is used to pull fluid across the flow cell. The resulting slide contains M13 template/oligo(dT) primer duplex. The temperature of the flow cell then is reduced to 37° C. for sequencing and the objective is brought into contact with the flow cell.

For sequencing, analogs of the invention (four species containing cytosine triphosphate, guanidine triphosphate, adenine triphosphate, or uracil triphosphate as the incorporatable base), each having a cyanine-5 label are stored separately in buffer containing 20 mM Tris-HCl, pH 8.8, 50 uM MnSO₄, 10 mM (NH₄)₂SO₄, 10 mM HCl, and 0.1% Triton X-100, and 100 U Klenow exo⁻ polymerase (NEB). Sequencing proceeds as follows.

First, initial imaging is used to determine the positions of duplex on the surface. The Cy3 label attached to the M13 templates is imaged by excitation using a laser tuned to 532 nm radiation (Verdi V-2 Laser, Coherent, Inc., Santa Clara, Calif.) in order to establish duplex position. For each slide only single resolvable fluorescent molecules imaged in this step are counted. Imaging of incorporated nucleotides as described below is accomplished by excitation of a cyanine-5 dye using a 635 nm radiation laser (Coherent). 100 nM Cy5CTP analog shown in FIG. 1 is placed into the flow cell and exposed to the slide for 2 minutes. After incubation, the slide is rinsed in 1×SSC/15 mM HEPES/0.1% SDS/pH 7.0 (“SSC/HEPES/SDS”) (15 times in 60 μl volumes each, followed by 150 mM HEPES/150 mM NaCl/pH 7.0 (“HEPES/NaCl”) (10 times at 60 μL volumes). An oxygen scavenger containing 30% acetonitrile and scavenger buffer (134 μl HEPES/NaCl, 24 μL 100 mM Trolox in MES, pH 6.1, 10 μL 100 nM DABCO in MES, pH 6.1, 8 μL 2M glucose, 20 μL 50 mM NaI (50 mM stock in water), and 4 μL glucose oxidase) is next added. The slide is then imaged (500 frames) for 0.2 seconds using an Inova310K laser (Coherent) at 647 nm, followed by green imaging with a Verdi V-2 laser (Coherent) at 532 nm for 2 seconds to confirm duplex position. The positions having detectable fluorescence are recorded. After imaging, the flow cell is rinsed 5 times each with SSC/HEPES/SDS (60 ul) and HEPES/NaCl (60 μL). Next, the cyanine-5 label is cleaved off incorporated CTP analog by introduction into the flow cell of 50 mM TCEP for 5 minutes, after which the flow cell is rinsed 5 times each with SSC/HEPES/SDS (60 μL) and HEPES/NaCl (60 μL).

The procedure described above then is conducted 100 nM Cy5dATP analog, followed by 100 nM Cy5dGTP analog, and finally 500 nM Cy5dUTP analog. The procedure (expose to nucleotide, polymerase, rinse, scavenger, image, rinse, cleave, rinse) is repeated as described above, except the UTP analog is incubated for 5 minutes instead of 2 minutes.

Once the desired number of cycles are completed, the image stack data (i.e., the single molecule sequences obtained from the various surface-bound duplex) are aligned to the M13 reference sequence. The alignment algorithm matches sequences obtained as described above with the actual M13 linear sequence. Placement of obtained sequence on M13 is based upon the best match between the obtained sequence and a portion of M13 of the same length, taking into consideration 0, 1, or 2 possible errors. All obtained 9-mers with 0 errors (meaning that they exactly match a 9-mer in the M13 reference sequence) are first aligned with M13. Then 10-, 11-, and 12-mers with 0 or 1 error are aligned. Finally, all 13-mers or greater with 0, 1, or 2 errors are aligned. Once complete, the sequence, including homopolymer counts is known.

### Example 5

In this example, three different nucleotide analogs of the invention were tested for their ability to be incorporated during a template-dependent sequencing reaction and to inhibit next base (or N+1) incorporation when sequencing through a homopolymer region. The nucleotide analogs were analyzed for their ability to be incorporated into the primer in a template-dependent fashion as well as their ability to be incorporated at the 3′ end of a primer at a rate comparable to that of a “non-blocked” analog. In addition, the nucleotide analogs were analyzed for their ability, once incorporated, to inhibit further base addition into the primer. In addition, the nucleotide analogs were analyzed to determine whether the inhibition was reversible upon removal of the blocking group.

The three different nucleotide analogs tested in this example include nucleotide analog 5 (as described in the Example 1), nucleotide analog 10 (shown below), and nucleotide analog 11 (also shown below).

Nucleotide analog 10 is set forth below:

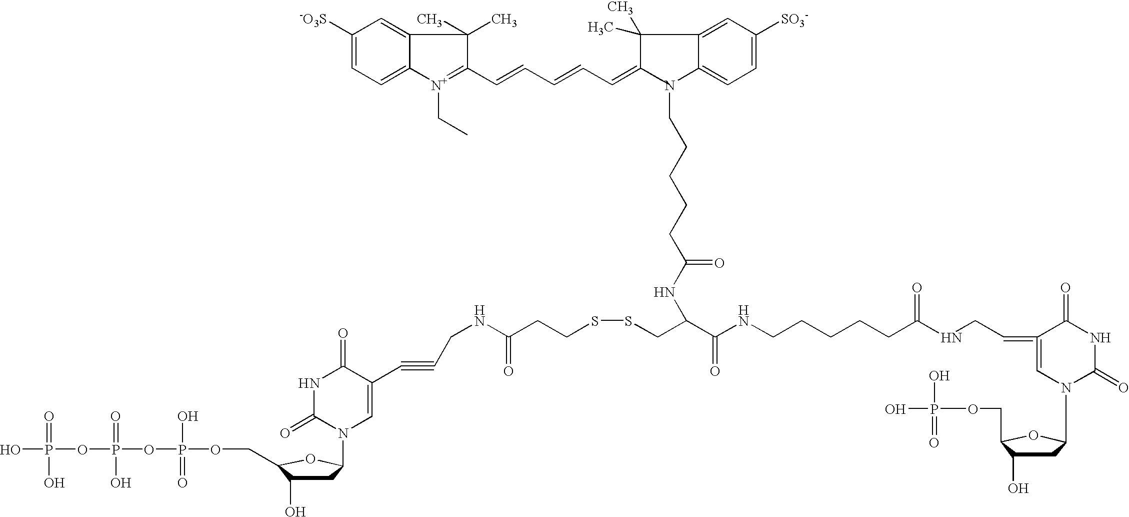

Nucleotide analog 11 is set forth below:

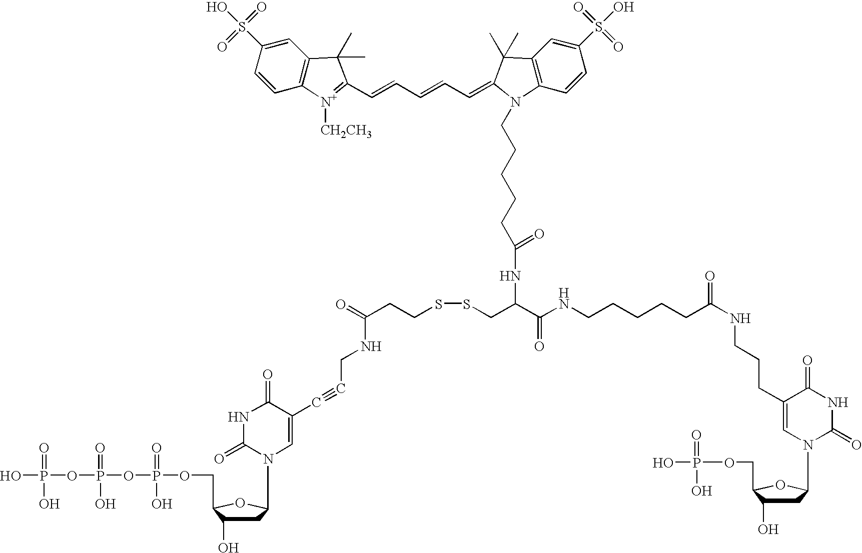

Each of the three nucleotide analogs were exposed to a T148 template containing a AAA homopolymer repeat with a primer hybridized to the template at a location proximal to the start of the homopolymer repeat. Each analog was presented at a concentration of 100 or 500 nM in the presence of Klenow exo⁻ under standard incorporation conditions as described previously. The rates of incorporation were compared to incorporation of a standard dUTP analog linked to a Cy5 dye via a 12 atom disulfide cleavable linker (referred to as dUTP-Cy5). The products of the reaction were analyzed using capillary electrophoresis. The results are presented below in Table 1.

TABLE 1

| | Nucleotide Analog | | |
|---|---|---|---|
| | Analog 5 | Analog 10 | Analog 11 |
| Rate of 1^{st} base (U) | 1.3× slower | Same as dUTP- | 1.2× slower |
| incorporation at start of | than dUTP- | Cy5 | than dUTP- |
| homopolymer region | Cy5 | | Cy5 |
| Rate of run-through to 2^{nd} | 70× slower | 110× slower | 143× slower |
| base (U) in homopolymer | than dUTP- | than dUTP-Cy5 | than dUTP- |
| region | Cy5 | | Cy5 |

As shown in Table 1, each of the exemplary nucleotide analogs was incorporated at a rate substantially the same as the dUTP-Cy5 analog but with substantially slower (70-150 fold slower) rate of incorporation of the second base. There was no detectable incorporation of any third base with any of the analogs.

Upon cleavage of the blocking group (left-hand portion of analog 5 (see Example 1) and the right-hand portion of the analogs 10 and 11 (see above)) by exposure to TCEP to cleave the disulfide bond, a new analog was added at a rate comparable to first base addition. These results show that the nucleotide analogs of the invention are incorporated during a template-dependent sequencing reaction and that they can significantly inhibit subsequent incorporation of a second base prior to removal of the blocking group.

### INCORPORATION BY REFERENCE

All publications, patents, and patent applications cited herein are hereby expressly incorporated by reference in their entirety and for all purposes to the same extent as if each was so individually denoted.

### EQUIVALENTS

While specific embodiments of the subject invention have been discussed, the above specification is illustrative and not restrictive. Many variations of the invention will become apparent to those skilled in the art upon review of this specification. Contemplated equivalents of the nucleotide analogs disclosed here include compounds which otherwise correspond thereto, and which have the same general properties thereof, wherein one or more simple variations of substituents or components are made which do not adversely affect the characteristics of the nucleotide analogs of interest. In general, the components of the nucleotide analogs disclosed herein may be prepared by the methods illustrated in the general reaction schema as described herein or by modifications thereof, using readily available starting materials, reagents, and conventional synthesis procedures. The full scope of the invention should be determined by reference to the claims, along with their full scope of equivalents, and the specification, along with such variations.

What is claimed is:

1. A nucleotide analog of Formula I or Formula II: 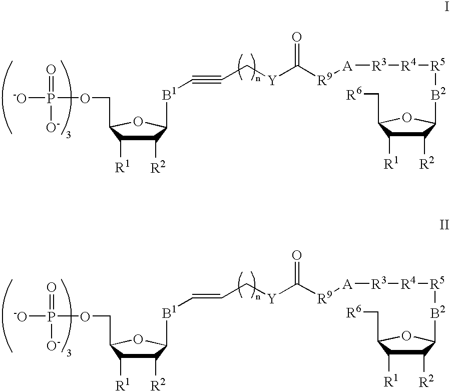
wherein,
B¹ and B² are each independently selected from the group consisting of a purine, a pyrimideine, and analogs thereof;
R¹ and R² at each occurrence are selected from the group consisting of OH, NH₂, F, N₃, and H;
Y is selected from the group consisting of NR′, O, S, CH₂, and a bond, wherein R′ is selected from the group consisting of H, alkyl, alkenyl, and alkynyl;
A is selected from the group consisting of —S—S—, an ester, and an amido group;
R³ is selected from the group consisting of: 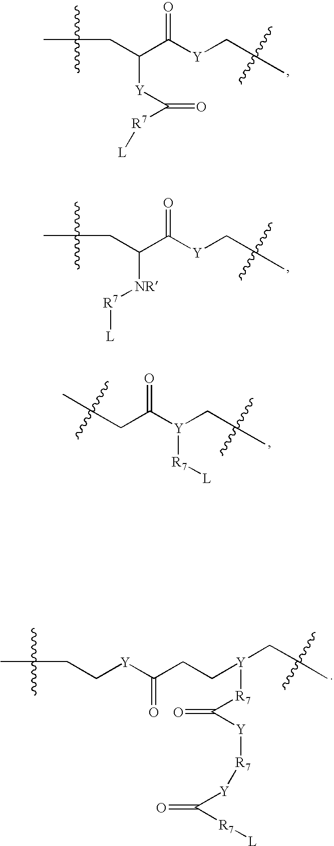
alkyl, and a bond;
R⁴ is selected from the group consisting of alkyl, alkenyl, alkynyl, ether, and a bond;
R⁵ is selected from the group consisting of: 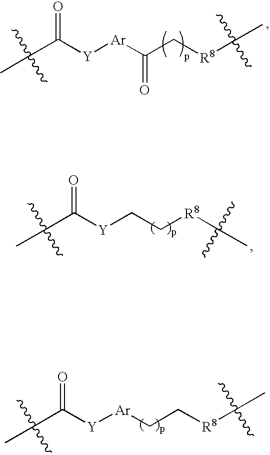
alkyl, alkenyl, and a bond;
Ar is aryl;
R⁶ is selected from the group consisting of: 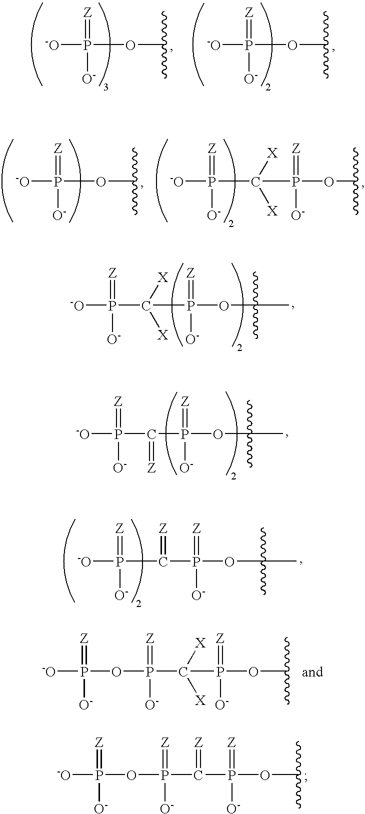
R⁷ is alkyl or a bond;
R⁸ is selected from the group consisting of S, alkyl, alkenyl, alkynl, and NR′;
R⁹ is selected from the group consisting of NR′, O, S, and —(CH₂)ₘ—;
L is a label;
X is H or a halogen;
Z, at each occurrence, independently, is O or S;
m, at each occurrence, independently is an integer from 0 to 50,
n, at each occurrence, independently is an integer from 0 to 50, and
p, at each occurrence, independently is an integer from 0 to 50.

2. The nucleotide analog of claim 1, wherein the double bond represented by 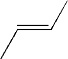
in Formula II is in a trans configuration.

3. The nucleotide analog of claim 1, wherein R⁶ is not

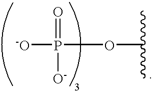

4. The nucleotide analog of claim 1, wherein R⁴ is glycol ether.

5. The nucleotide analog of claim 1, wherein Ar is phenyl or aromatic acid.

6. The nucleotide analog of claim 1, wherein n is 1 or 4.

7. The nucleotide analog of claim 1, wherein R⁹ is —(CH₂)ₘ—.

8. The nucleotide analog of claim 7, wherein n is 4 and m is 3.

9. The nucleotide analog of claim 7, wherein m is 0, 2 or 3.

10. The nucleotide analog of claim 1, wherein R¹ is OH and R² is H.

11. The nucleotide analog of claim 1, wherein B and B are each independently selected from the group consisting of cytosine, uracil, thymine, adenine, guanine, and analogs thereof.

12. The nucleotide analog of claim 1, wherein L is an optically detectable label.

13. The nucleotide analog of claim 12, wherein the optically detectable label is a fluorescent label.

14. The nucleotide analog of claim 13, wherein the optically detectable label is selected from the group consisting of cyanine, rhodamine, fluoroscein, coumarin, BODIPY, Alexa and conjugated multi-dyes.

15. The nucleotide analog of claim 13, wherein the fluorescent label is Cy3 or Cy5.

16. The nucleotide analog of claim 1, wherein when R³ is alkyl or a bond, L is covalently bonded to R¹, R², R⁵, R⁶ or B².

17. The nucleotide analog of claim 16, wherein L is covalently attached to R¹ or R² via an amide linkage.

18. The nucleotide analog of claim 17, wherein the amide linkage is —CH₂—S—S—CH₂—CH₂—NHCO—.

19. The nucleotide analog of claim 1, wherein L is covalently attached to R⁵ or R⁶ via an amide bond.

20. A nucleotide analog represented by: 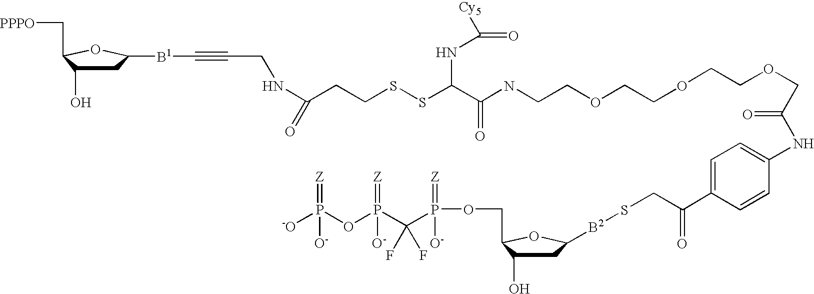
wherein B¹ and B² are each independently selected from the group consisting of cytosine, uracil, thymine, adenine, guanine, and analogs thereof;
PPPO— is 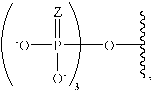
and
Z, at each occurrence, independently is O or S.

21. A nucleotide analog of represented by Formula III: 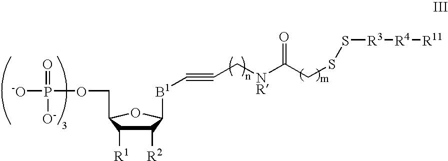
wherein
R¹¹ is selected from the group consisting of: 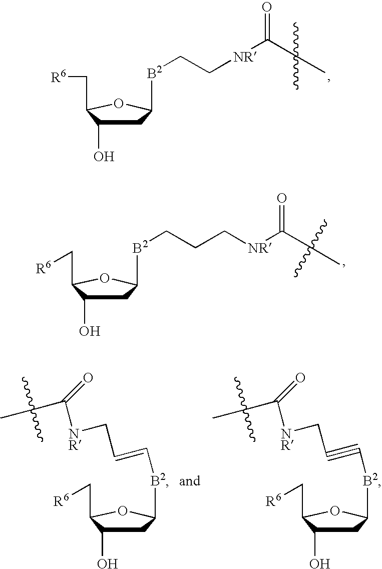
wherein B¹, B², Y, R′, Z, L, R¹, R², R³, R⁴, R⁶, R⁷, m and n, are as defined in claim 1.

22. The nucleotide analog of claim 21 wherein B¹ and B² are each independently selected from the group consisting of cytosine, uracil, thymine, adenine, guanine, and analogs thereof.

23. The nucleotide analog of claim 21, wherein L is an optically detectable label.

24. The nucleotide analog of claim 23, wherein the optically detectable label is a fluorescent label.

25. The nucleotide analog of claim 24, wherein the optically detectable label is selected from the group consisting of cyanine, rhodamine, fluoroscein, coumarin, BODIPY, alexa and conjugated multi-dyes.

26. The nucleotide analog of claim 24, wherein the fluorescent label is Cy3 or Cy5.

27. The nucleotide analog of claim 21, wherein when R³ is alkyl or a bond, and L is covalently bonded to R¹¹.

28. A nucleotide analog selected from the group consisting of: 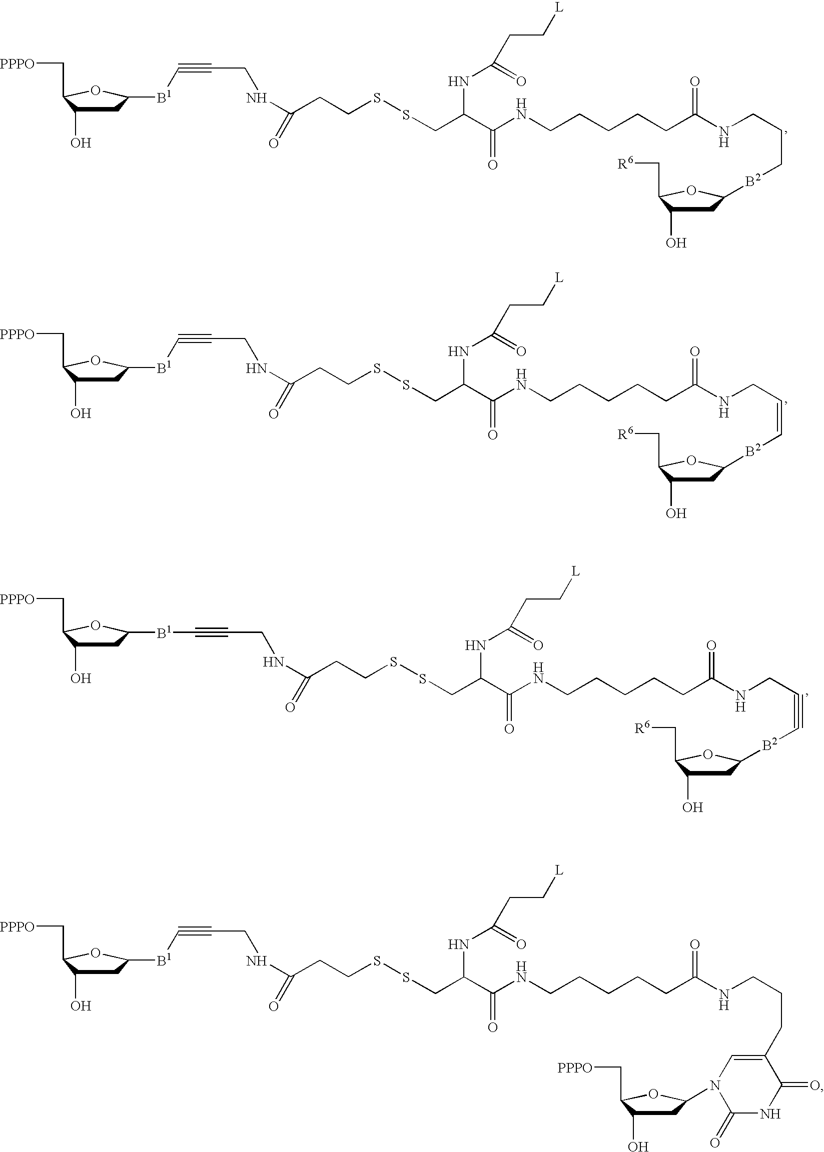 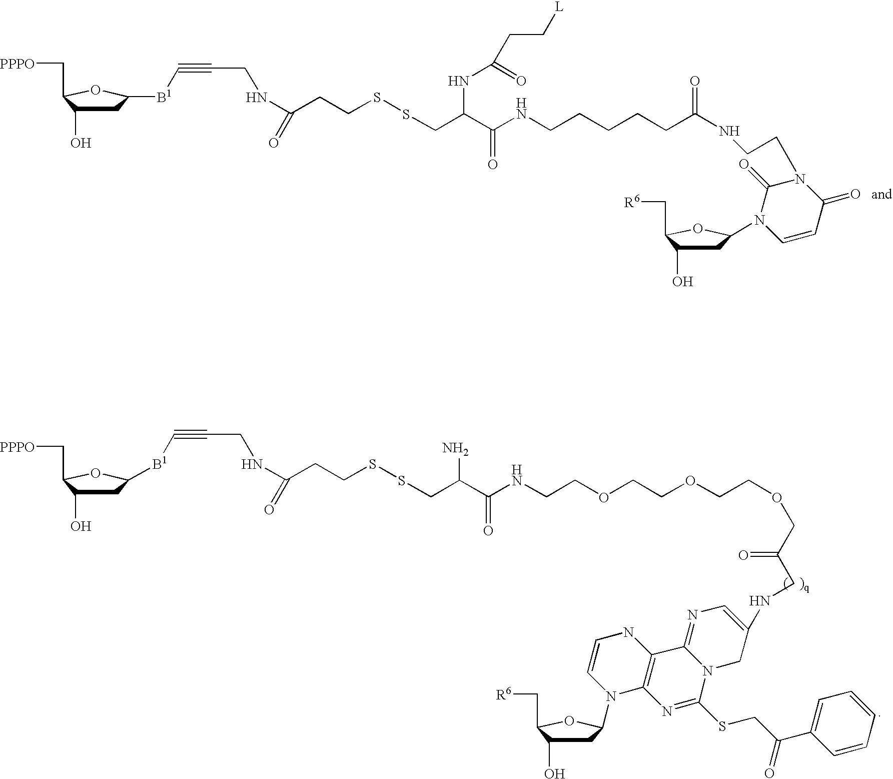
wherein, in each structure, B¹, B², R⁶, and L are as defined in claim 1, q is an integer from 1 to 50, PPPO— is 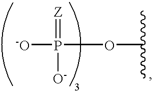
and Z, at each occurrence, independently is O or S.

29. The nucleotide analog of claim 28, wherein L is a fluorescent label.

30. The nucleotide analog of claim 29, wherein the fluorescent label is selected from the group consisting of Cy5, Cy3, rhodamine, fluoroscein, coumarin, BODIPY, alexa and conjugated multi-dyes.

31. A nucleotide analog of Formula IV or Formula V: 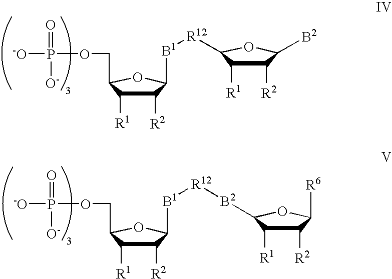
wherein B¹, B², R¹ and R² as defined in claim 1;
R¹² represents a moiety comprising a cleavable linker; and
R⁶ represents any moiety with the proviso that R⁶is not

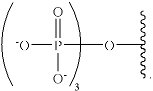

32. The nucleotide analog of claim 31, wherein R¹² comprises an alkynl moiety bound to B¹.

33. The nucleotide analog of claim 31, wherein R¹² comprises an alkynl moiety bound to B².

34. The nucleotide analog of claim 31, wherein R⁶ is selected from the group consisting of: 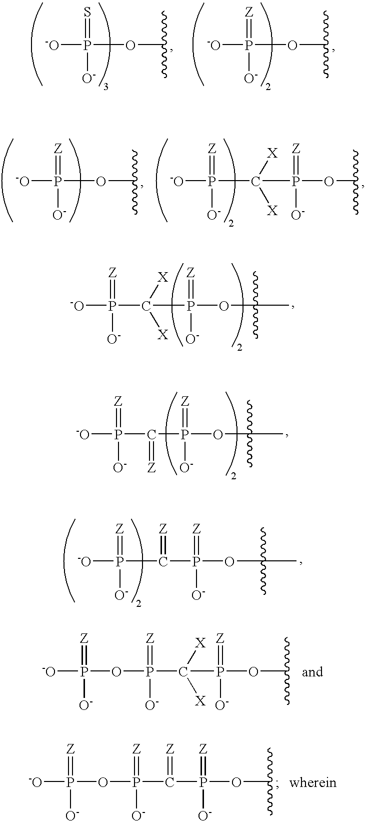
X is H or a halogen;
Z, at each occurrence, independently, is O or S

35. A method of sequencing a nucleic acid template comprising:
(a) exposing a nucleic acid template hybridized to a primer having a 3′-OH end to (i) a polymerase which catalyzes nucleotide additions to the primer, and (ii) the nucleotide analog as shown in claims 1-34 under conditions to permit the polymerase to add the nucleotide analog to the primer;
(b) detecting the nucleotide analog added to the primer in step (a);
(c) removing the label from the nucleotide analog; and
(d) repeating steps (a), (b) and (c) thereby to determine the sequence of the template.

36. A method of sequencing a nucleic acid template comprising:
(a) exposing a nucleic acid template comprising first and second consecutive bases that is hybridized to a primer having a 3′ end to (i) a polymerase which catalyzes nucleotide additions to the primer, and (ii) a labeled nucleotide analog comprising a first nucleotide or a first nucleotide analog covalently bonded through a linker to a blocking group, under conditions to permit the polymerase to add the labeled nucleotide analog to the primer at a position complementary to the first base while preventing another nucleotide or nucleotide analog from being added to the primer at a position complementary to the second base;
(b) detecting the nucleotide analog added to the primer in step (a);
(c) removing the blocking nucleotide or blocking nucleotide analog; and
(d) repeating steps (a), (b) and (c) to determine the sequence of the template.

37. The method of claim 36, wherein the blocking group is a second nucleotide analog or a second nucleotide analog.

38. The method of claim 37, wherein the linker is covalently attached to the base of the first nucleotide or first nucleotide analog and to the base of the second nucleotide or second nucleotide analog.

39. The method of claim 38, wherein the linker contains from about 4 to about 50 atoms.

40. The method of claim 38, wherein the linker contains from about 15 to about 50 atoms.

41. The method of claim 36, wherein the linker is covalently attached to the first nucleotide or first nucleotide analog via an alkynyl group or an alkenyl group containing a double bond in a trans configuration.

42. The method of claim 36, wherein, in step (a), the labeled nucleotide analog comprises a nucleotide analog of claim 1, 20, 21, 28 or 31.

43. The method of claim 36, wherein, in step (b), the label is removed at the same time as the blocking group.

44. The method of claim 36, wherein the label is an optically detectable label.

45. The method of claim 36, wherein the conditions are sufficient to detect and sequence single molecules individually.

46. An improved method of sequencing a nucleic acid template containing a homopolymer region using a primer complementary to at least a portion of the template and a polymerase, wherein the improvement comprises performing each cycle of a sequencing reaction in the presence of a labeled nucleotide analog comprising a first nucleotide or first nucleotide analog covalently bonded through a linker to a blocking group under conditions to cause the polymerase to add only a single labeled nucleotide analog to a chain extending from the primer at a position complementary to one base in the homopolymer region.

47. The method of claim 46, wherein the blocking group is a nucleotide or a nucleotide analog.
